(19) United States
(12) Patent Application Publication
(10) Pub. No.: US 2020/0118661 A1
(43) Pub. Date: Apr. 16, 2020

(54) COMMUNICATION CHANNEL AGGREGATION WITH MONITORING USING A SOFTWARE MEDIATOR

(71) Applicant: Pyx Health, Inc., Phoenix, AZ (US)

(72) Inventors: Cynthia K. Jordan, Tucson, AZ (US); Michael Patrick Martin, Tucson, AZ (US)

(21) Appl. No.: 16/588,303

(22) Filed: Sep. 30, 2019

Related U.S. Application Data
(60) Provisional application No. 62/745,871, filed on Oct. 15, 2018.

(51) Int. Cl. G16H 20/00; G16H 50/30; G16H 80/00; H04W 4/021

(52) U.S. Cl. CPC G16H 20/00; H04W 4/021; G16H 80/00; G16H 50/30

(57) ABSTRACT

Systems for aggregating communication channels to generate effective interventions is provided. Some such systems include an intervention mediator configured to electronically communicate with at least one of a first user via a first user device and a second user via a second user device. The mediation module is configured to assess whether an intervention with the first user is appropriate, determine a specific intervention based at least in part on a result of the assessment, and provide the second user, via the second user device, a prompt to perform the specific intervention for the benefit of the first user. Methods for selectively generating a natural support intervention are also provided.

### CROSS-REFERENCE TO RELATED APPLICATIONS

This application claims priority to U.S. Provisional Application Ser. No. 62/745,871, filed Oct. 15, 2018, which is incorporated by reference in its entirety herein.

### BACKGROUND

### Field

The present disclosure relates to communication channel aggregation. Specifically, the present disclosure relates to communication channel aggregation and monitoring using a software mediator.

### Related Technology

Current health care models provide needed medical and health care to patients. However, such current health care models rely on and require a plurality of separate communication paths between the caregiver and the patient, especially between doctor's visits. For example, a patient may visit the doctor in person to obtain initial screening and care. Follow ups may occur through different communication channels. For example, a doctor and/or a third-party caregiver may telephone the patient with additional information and/or instructions. The doctor, third-party caregiver and/or patient may further communicate through email, text or multi-media message, or even through physical mail. However, each of these modes of communication utilize a different system of communication and monitoring patient care across these many and varied modes of communication can become difficult for the doctor, any caregivers, and the patient. In some cases, this multiplicity of communication channels and the associated difficulty can lead to reduced compliance and/or engagement of the patient with their health plan, health providers, caregivers and/or doctors.

### SUMMARY

According to some embodiments, a system for selectively generating a prompt for an intervention is provided. The intervention prompts are mediated by a software intervention mediator that may take the form of a virtual persona.

### BRIEF DESCRIPTION OF THE DRAWINGS

FIG. 1A shows an example of a system for selectively generating a natural support intervention, according to some embodiments.

FIG. 1B shows an example of the system of FIG. 1A in more detail, according to some embodiments;

FIG. 2 is a diagram illustrating several structural and/or functional aspects of the system of FIGS. 1A and 1B, according to some embodiments;

FIG. 3 illustrates a flowchart of a method for selectively generating a natural support intervention for a user, in accordance with some embodiments;

FIG. 4 illustrates a flowchart of another method for selectively generating a natural support intervention for a user, in accordance with some embodiments.

FIG. 5 illustrates a flowchart of a method for selectively generating a geolocation-based natural support intervention for a user, in accordance with some embodiments;

FIG. 6 illustrates several example screen-shots for an application configured to facilitate natural support intervention for a user, in accordance with some embodiments;

FIG. 7 illustrates several additional example screen-shots for an application configured to facilitate natural support intervention for a user, in accordance with some embodiments;

FIG. 8 illustrates several additional example screen-shots for an application configured to facilitate natural support intervention for a user, in accordance with some embodiments;

FIG. 9 illustrates several additional example screen-shots for an application configured to facilitate natural support intervention for a user, in accordance with some embodiments;

FIG. 10 illustrates several additional example screen-shots for an application configured to facilitate natural support intervention for a user, in accordance with some embodiments;

FIG. 11 illustrates several additional example screen-shots for an application configured to facilitate natural support intervention for a user, in accordance with some embodiments;

FIG. 12 illustrates several additional example screen-shots for an application configured to facilitate natural support intervention for a user, in accordance with some embodiments; and

FIG. 13 illustrates several additional example screen-shots for an application configured to facilitate natural support intervention for a user, in accordance with some embodiments.

### DETAILED DESCRIPTION

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any number of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different wireless technologies, system configurations, networks, and transmission protocols, some of which are illustrated by way of example in the figures and in the following description of the preferred aspects. The detailed description and drawings are merely illustrative of the disclosure rather than limiting, the scope of the disclosure being defined by the appended claims and equivalents thereof.

### Definitions

App: An app is a software program that is capable of executing on smartphone operating systems such as iOS and Android, also referred to as a mobile app, although it can be executed on non-mobile devices that are running an appropriate operating system.

Bot: A bot is a software program that performs automated functions with Internet resources in response to inputs. The inputs may be human interactions with a user interface such as a touchscreen, a keyboard, or a microphone. In many applications, at least some of the automated functions include text or speech outputs delivered in response to inputs.

Geofence: A geographic area or boundary defined in an app. The app may monitor the location of a device executing the app using location determination capabilities of the device, and the app may execute a defined function when the app determines that the device is inside or outside the defined area or boundary.

Browser engine and Web Page: A browser engine is a computer program that provides functionality to a computer for executing syntax contained in web pages. The computer may be connected to a computer network, and the network may be, and usually will be, the Internet. As used herein, browser engines and web pages together provide functionality to a computer connected to a network (e.g. the Internet) at least sufficient to request, retrieve, and display at least some network resources including web pages themselves, and to execute at least some links contained within or referred to in retrieved web pages to retrieve other web pages specified with the links. Web pages may include references such as uniform resource locators (URLs) and/or universal resource identifiers (URIs) to other network resources that contain images or other data that is retrieved by the browser engine from the network or from a cache memory when executing the web page, and may also include references to programs, libraries, style sheets, scripts, and the like which are called by the browser engine when executing the web page. Some of these items may require prior separate loading onto the computer (e.g. Flash and a Java Virtual Machine). Any of these items that are accessed, used, and/or retrieved during browser engine execution of web page syntax are considered to be included as a component of the “web page” as that term is used herein. Examples of browser engines include, but are not limited to, Internet Explorer and Edge distributed by Microsoft, and Chrome distributed by Google. Example web page syntax that can be executed by browser engines is the various versions of HyperText Markup Language (HTML) promulgated by the World Wide Web Consortium (W3C).

Browser Extension: A computer program that adds functionality to a browser engine but is distributed separately from the browser engine to which it adds that functionality. Popular browser engine programs such as Internet Explorer and Chrome provide internal functionality allowing them to interact with browser extensions distributed by third parties as long as the third-party browser extension complies with the interface for browser extensions provided with the browser engine. The functionality provided by a browser extension may include modifications to browser execution of retrieved web pages. Some currently available browser extensions perform functions such as displaying and executing toolbar functions on top of retrieved web pages or blocking browser retrieval of advertisements that would otherwise be downloaded in conjunction with web page execution. In some embodiments, a browser extension will be loaded and executed within designated browser engine memory anytime the browser engine is loaded into the browser engine memory. Accordingly, in some embodiments, such a browser extension may be periodically, intermittently, or continuously monitoring operations of the browser engine and, in some cases as will be described in more detail below, intercepting one or more communications from and/or to the browser engine.

Plug-in: In contrast to a browser extension, a plug-in is a computer program invoked by the browser engine, and run in browser engine memory, specifically in response to the browser engine reading code that invokes the plug-in. Accordingly, a browser engine may operate for extended periods of time without invoking and/or loading a particular plug-in and a plug-in never intercepts communications not addressed to the plug-in. Moreover, plug-ins must be explicitly loaded and/or enabled by a user of a browser engine in response to the browser engine reading code requiring functionality of the plug-in. For example, the Adobe Flash plug-in provides functionality to a browser engine for decoding and rendering Flash video files in response to the browser engine reading at least a portion of a Flash video file and further requires the user to explicitly download and install the Flash plug-in, ensuring the viewing experience is not seamless or transparent to the user.

Server: Processing hardware coupled to a computer network having network resources stored thereon that is configured with software to respond to client access requests to use or retrieve the network resources stored on the server.

Internet: The globally interconnected system of computers and computer networks that evolved from ARPANET and NSFNET over the late 1980s and early 1990s that may utilize TCP/IP network communication protocols.

Network Resource Identifier: A definition of a network resource (e.g. by storage location and filename) that is used by client computers to specify a network resource in access requests issued to the network by the client computers. A network resource identifier may also be referred to as a location of a network resource such as an image or a web page. Currently, when the network is the Internet, Network resource identifiers are known as URLs that are formatted in accordance with RFC 3986 of the Internet Engineering Task Force (IETF). For the purposes of this disclosure, any format for specifying a network resource in client access requests issued to a network is within the definition of the term Network Resource Identifier. A network resource identifier, including URLs as currently defined on the Internet, may further include data in addition to data identifying the network resource that a server hosting the network resource associated with the network resource identifier may use for other purposes beyond identifying the requested network resource.

Web Site: A collection of network resources including at least some web pages that share a common network resource identifier portion, such as a set of web pages with URLs sharing a common domain name but different pathnames.

Web Server: A server that includes functionality for responding to requests issued by browsers to a network, including, for example, requests to receive network resources such as web pages. Currently, browsers and web servers format their requests and responses thereto in accordance with the HyperText Transfer Protocol (HTTP) promulgated by the IETF and W3C. In some embodiments, a web server may also be a content server.

World Wide Web: The collection of web pages stored by and accessible to computers running browsers connected to the Internet that include references to each other with links.

Link: Syntax that instructs a browser executing the syntax to access a network resource defined directly or indirectly by the syntax. The link syntax and/or internal browser engine functionality may also define conditions under which the access request is made by the browser engine, for example through cursor position and/or other interaction with an I/O device such as a keyboard or mouse. Some link syntax may cause the browser engine to access the specified network resource automatically while processing the syntax without user prompt or interaction. Links include HTML hyperlinks. A link may be directly coded with, for example, HTML tags and an explicit URL, or may be in the form of a script or other called function defined in a browser, in a browser extension, and/or in a webpage.

Network Resource: A web page, file, document, program, service, or other form of data or instructions which is stored on a network node and which is accessible for retrieval and/or other use by other network nodes.

Redirection Response: A response that may be provided by a server when processing an access request of a client for a network resource, where the response includes a network resource identifier of a different network resource that the client should access for the desired information or action. In the HTTP protocol, a redirection response may also include a 303 status code, and the client receiving the redirection response may then send a GET or other request for the network resource identified by the URL provided in the response.

Navigate: Controlling a browser engine and/or a browser extension so as to use a series of links to access a series of network resources.

Several embodiments will now be described in connection with one or more of the figures below.

The present disclosure is directed to a user network that utilizes a software application that may advantageously be a mobile app. The software may instantiate an intervention mediator that may take the form of a bot that functions as a virtual mediation persona interacting with both a first user and a second user over the network to thereby facilitate and mediate consolidated communication and/or interaction between the first user and the second user through the software. By facilitating, mediating, and prompting communication and interaction between the first user and the second user through the software, the number of different communication channels between the first and second users may be substantially reduced.

In some embodiments, such a network and app may be particularly advantageous when utilized in a caregiving environment, although the present disclosure is not limited to such environments. For example, the first user may be a patient and the second user may be a caregiver, for example, a doctor, a family member or a third-party caregiver of the patient. In such a context, natural support intervention and self-management of patient care may be more easily obtained for a patient. This provides a user-centered solution that leverages chatbot technology, a user's natural support network, and real-time alerts to provide informed, relevant, and non-clinical support to the user by harnessing the power of the most influential people in the user's life—their friends and family—to encourage self-management and drive improved health outcomes. As will be described in more detail in the following description, a virtual mediation persona “walks” alongside the user in their health care journey and keeps their support network engaged, through interaction with the app, and helps the user's friends and family gain actionable insights and encourages real-time intervention with the user.

As a majority of healthcare happens between provider visits, this space between visits is critically important as it relates to outcomes and user satisfaction. The present disclosure provides systems, networks and methods for engaging users within that space between provider visits to gain powerful insights and capture data critical to desirable healthcare outcomes. Advantages may include but are not limited to reduced readmissions, better understanding and addressing care outside of the clinical setting, preventions of health crises for users, leveraging real-time data to drive decisions around care planning, engaging user's natural supports to improve health outcomes, and meeting user's individual needs in a scalable way. Additional advantages may include but are not limited to enhanced care coordination between the user's health plan, natural and peer community supports, self-management through evidence-based screening tools and proprietary algorithms, tracking of social determinants of health to proactively identify when a user needs assistance and provide real-time resources to the user, instant notification of when users utilize healthcare, emergency room/urgent care/crisis care, social or community services, provision of access to real-time intelligence into the user's mood, wellness and activity levels for support network and health plan partners, and support network scalability.

Examples of users who may find such a network advantageous include but are not limited to seniors who are lonely or sick, high-risk pregnant women, transition-age youth, users receiving behavior health services, users suffering from chronic pain, high-touch Care Management “graduates,” and users who are suffering from homelessness. Entities that may find such a network advantageous include but are not limited to health plan organizations, Medicaid payers, Medicare payers, self-insured employers and/or partners of health plans.

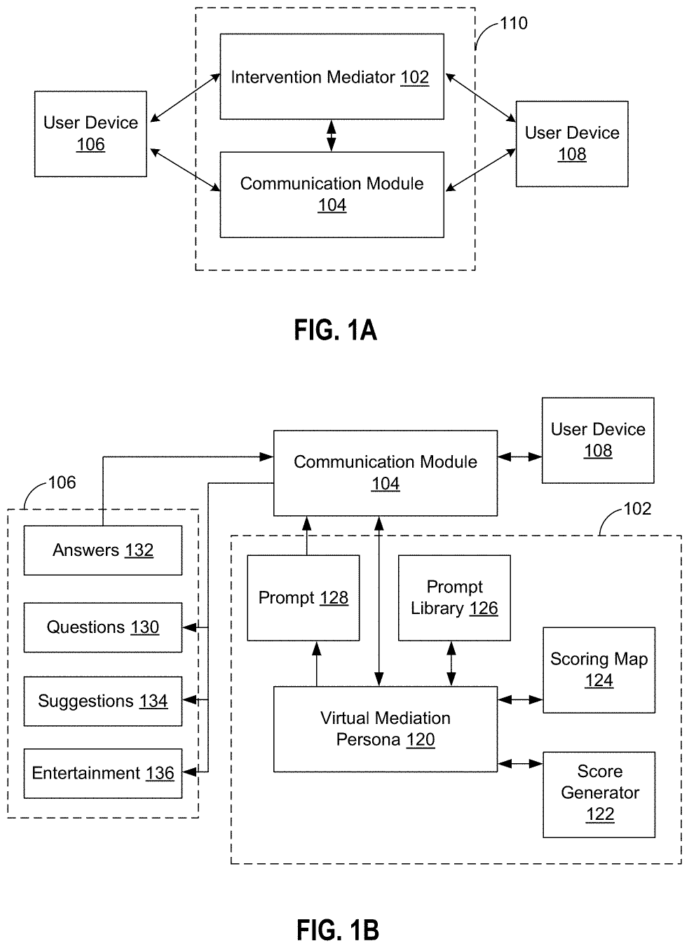

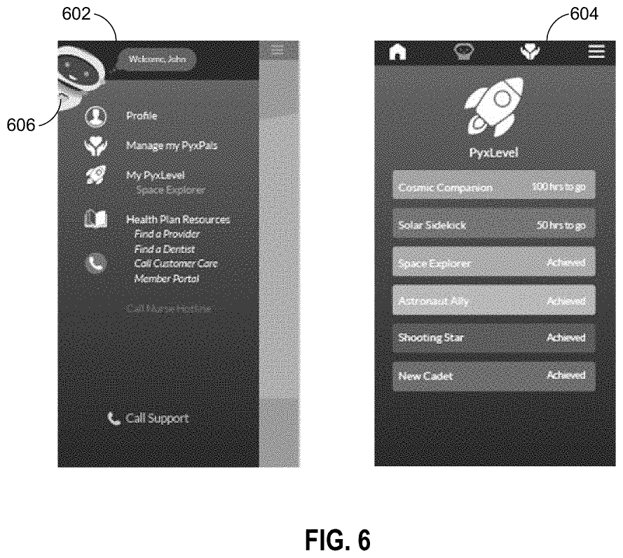

FIG. 1A shows an example of a system 110 for selectively generating an intervention, according to some embodiments. System 110 comprises an intervention mediator 102 and a communication module 104, each configured to communicate with one another as well as with a first user via an app on user device 106 and a second user via an app on user device 108. In some embodiments, intervention mediator 102, or one or more components of intervention mediator 102, may also be described as a mediator. In some embodiments, the first user may be a patient who interacts with system 110 via user device 106, which may be a smartphone, personal computer, or any other electronic device configured to communicate with system 110, for example. The second user may be a caregiver, family member or doctor who interacts with system 110 and ultimately with the first user through system 110 via user device 108, which may be a smartphone, personal computer, or any other electronic device configured to communicate with system 110. In some cases, the user device may be dedicated to communicating with the user and the system 110, for example, it may be a stand-alone device that sits or is attached to a bedside table. As will be described in more detail in the following description, the first user's interaction with intervention mediator 102 via the app on user device 106 drives interaction between intervention mediator 102 and either or both of the first user via the app on user device 106 and the second user via the app on user device 108, thereby facilitating natural support intervention and self-management of patient care for the first user. Such interaction between the first user and system 110, as well as interaction between the second user and system 110 may be mediated by a virtual mediation persona (VMP or bot) that appears on user devices 106, 108 and interacts with the users (see VMP 606 illustrated in at least FIGS. 6, 9 and 13). Accordingly, at least a portion of communications and/or interactions between the first user and the second user comprise interactions between the first user and VMP 606 via the app on user device 106 and separate interactions between VMP 606 and the second user via the app on user device 108.

FIG. 1B shows an example of system 110 of FIG. 1A in more detail, according to some embodiments. Intervention mediator 102 comprises a virtual mediation persona module 120 configured to generate an interaction with the first user via the app on user device 106 to assess a physical, mental or emotional state of the first user and determine whether a natural support intervention is appropriate. In some embodiments, such an interaction comprises virtual mediation persona module 120 generating one or more questions 130 regarding the mood, wellness and/or activity of the first user and VMP 606 asking or presenting questions 130 to the first user, for example utilizing communication module 104. In some embodiments, virtual mediation persona module 120 is configured to customize questions 130 through machine learning, based on one or more cognitive services, and/or based on a predetermined script or flowchart and particular answers provided by the first user. In some other embodiments, questions 130 are selected from a questions library.

Virtual mediation persona module 120 may be configured to receive, via communication module 104, one or more answers 132 to questions 130 from the first user, entered via the app on user device 106. Utilizing answers 132, virtual mediation persona module 120 may be configured to generate an intervention score for determining whether a particular natural support intervention is appropriate utilizing a scoring module 122. Virtual mediation persona module 120 may be configured to map the generated score to one or more interactions with natural support utilizing a scoring map 124. Based on a mapping of the generated score to one or more interactions with natural support, virtual mediation persona module 120 may select one or more prompts 128 associated with the one or more interactions with natural support from a prompt library 126 and deliver one or more of prompts 128 to user device 106. In some embodiments, the one or more prompts 128 may comprise one or more suggestions 134 and/or entertaining interactions 136 with the first user, for example follow-up intervention chatting, which, in some cases, is mediated or presented to the first user by VMP 606.

Virtual mediation persona module 120 may be further configured to send one or more of prompts 128, in some cases different from the one or more prompts 128 sent to the first user, to user device 108, via communication module 104, thereby suggesting one or more interventions for the second user to perform with respect to the first user. For example, the second user may be prompted by VMP 606 within the app on user device 108 to contact the first user via chat asking how the first user is feeling. The first and second users may then chat, video conference, or otherwise interact with one another via their respective apps on user device 106 and user device 108 via communication module 104. Accordingly, not only are natural support interventions between the first and second users facilitated, the communication channels by which such natural support interventions are facilitated are aggregated and/or consolidated to communications via system 110, through communication module 104.

The functionality of system 110, and in some cases intervention mediator 102 and/or virtual mediation persona module 120 therein, will now be described in more detail in connection with the following figures.

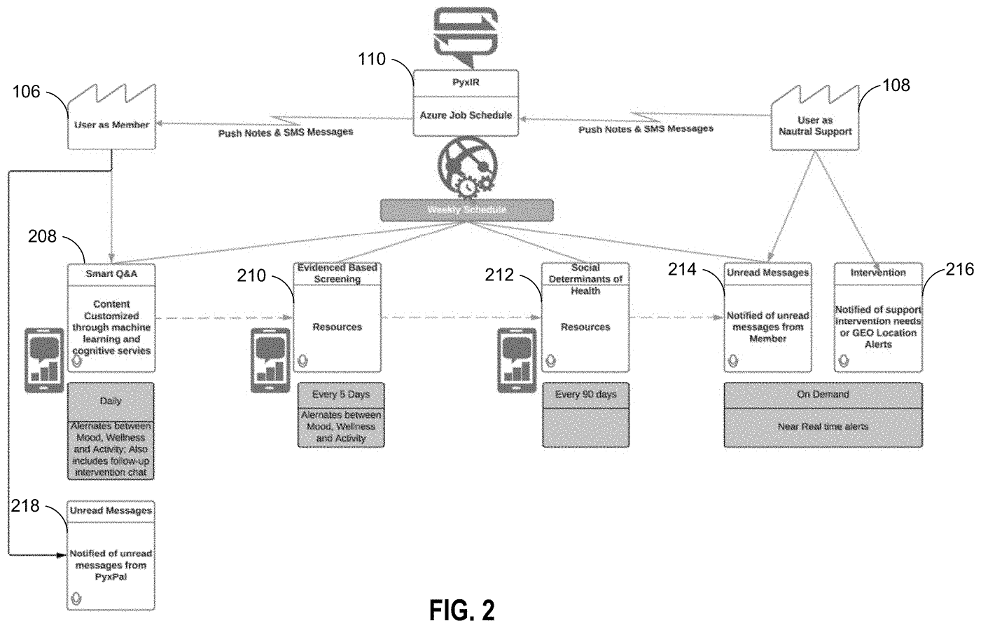

FIG. 2 is a diagram illustrating several structural and/or functional aspects of system 110 of FIGS. 1A and 1B, according to some embodiments. System 110 is illustrated as being communicatively coupled to user device 106 and to user device 108. System 110 may be configured to send and receive any communications as described in this disclosure to user device 106 and user device 108. Further functionality of system 110, and in some cases more specifically virtual mediation persona module 120 of intervention mediator 102, will be described below.

System 110 may be configured to perform a smart question and answer session 208 on a repeating, periodic, or random basis (for example daily), which includes providing a set of questions to user device 106, which may alternate between topics including but not limited to the mood, wellness and/or activity of the first user. System 110 may receive the answers to the set of questions and provide follow-up intervention chat between the first user and VMP 606. System 110 and/or user device 106 may also be configured to provide a notification 218 to the first user of any unread messages or interactions with the first user.

System 110 may be configured to periodically or randomly perform an evidence-based screening 210 (for example every 5 days) utilizing the answers provided by the first user via user device 106 and provide one or more resources for self-management and/or natural support intervention to one or both of the first and second users via user devices 106, 108.

System 110 may be configured to periodically or randomly evaluate social determinants of health 212 (for example every 90 days) utilizing the answers provided by the first user via user device 106 and provide one or more resources for self-management and/or natural support intervention to the first user via user device 106 and/or to the second user via user device 108. For example, if something negative occurs socially for the first user, or they or someone in their family or support group moves, there can be medical consequences to the psychological state caused by such occurrences. By evaluating answers 132 and, in some cases, determining further appropriate questions 130 based on answers 132, such social determinants of heath 212 may be detected and self-management and/or natural support interventions may be prompted to thereby relieve or reduce any associated negative health consequences.

System 110 may be configured to provide to the second user, on demand in real-time or near real-time via user device 108, any of the information provided to or received from user device 106 and/or any results, evaluations, resources, interventions or other events determined by system 110 such that the second user may monitor and effectively provide natural support intervention to the first user as needed. For example, and not limitation, system 110 may provide a notification 214 to the second user of any unread messages for the first user or for the second user. System 110 may also provide a notification 216 of any support intervention needs of the first user or geolocation alerts, as will be described in more detail in the following description.

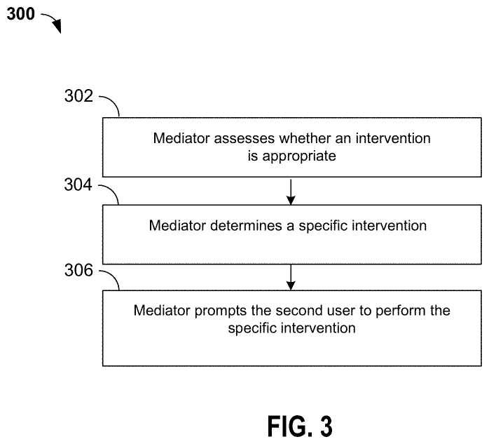

FIG. 3 illustrates a flowchart 300 of a method for selectively generating a natural support intervention for a user, in accordance with some embodiments. In some embodiments, the method of flowchart 300 may be performed by system 110, intervention mediator 102, or virtual mediation persona module 120, as previously described in connection with FIGS. 1A-2, any of which may be referred to as a mediator herein. In some embodiments, some or all of the described interactions with the first user and/or the second user may be mediated by VMP 606.

At block 302, the mediator assesses whether a natural support intervention is appropriate. For example, system 110, intervention mediator 102, or virtual mediation persona module 120 may provide questions 130 to the first user via the app on user device 106, receive answers 132 from the first user via the app on user device 106, and utilizing scoring module 112, generate a score that may be used to determine an appropriate natural support intervention.

At block 304, the mediator determines an appropriate natural support intervention. For example, system 110, intervention mediator 102, or virtual mediation persona module 120 may utilize scoring map 124 to map the generated score to the appropriate natural support intervention.

At block 306, the mediator prompts at least one of the first user and the second user to perform the appropriate natural support intervention. For example, system 110, intervention mediator 102, or virtual mediation persona module 120 may send a first natural support intervention to user device 106 such that the first user is notified and encouraged to perform the first natural support intervention. Examples of such a first intervention or care self-management may include but are not limited to initiating a chat or video conference session with the second user, who may be a family member, friend, doctor or caregiver, going for a walk, or performing some other task or activity expected to improve a state of the first user. In addition, or in the alternative, the mediator may send a second natural support intervention to user device 108 such that the second user is notified and encouraged to perform the second natural support intervention. Such a prompt may be delivered in a form of daily insights into the past or current state of the first user. Examples of such a second natural support intervention may include but are not limited to initiating a chat or video conference session with the first user, who may be the patient, meeting up with the first user to socialize or to check in on his or her condition, or performing some other task or activity expected to improve the state of the first user.

Such prompted interventions, regular contact or communication for natural support intervention between the first and second users via user devices 106, 108 may be facilitated through the mediator, such that all electronic communications occur through system 110 and are thereby consolidated or aggregated.

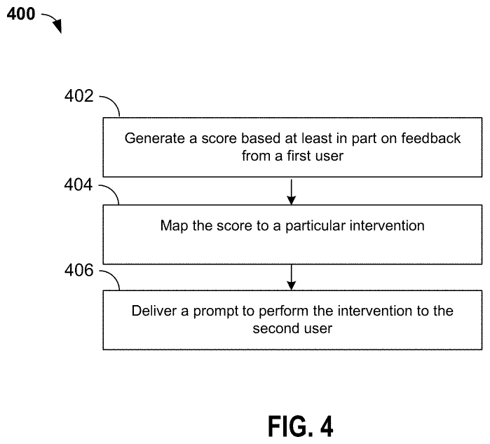

FIG. 4 illustrates another method for selectively generating a natural support intervention for a user, in accordance with some embodiments. In some embodiments, the method of flowchart 400 may be performed by system 110, intervention mediator 102, or virtual mediation persona module 120, as previously described in connection with FIGS. 1A-2, any of which may be referred to as a mediator herein. In some embodiments, some or all of the described interactions with the first user and/or the second user may be mediated by VMP 606.

Block 402 includes generating a natural support intervention score based at least in part on feedback from a first user. For example, virtual mediation persona module 120 may provide questions 130 to the first user via the app on user device 106, receive answers 132 from the first user via the app on user device 106, and utilize scoring module 122 to generate a natural support intervention score based at least in part on answers 132.

Block 404 includes mapping the score to a natural support intervention. For example, virtual mediation persona module 120 may utilize scoring map 124 to map the generated score to an appropriate intervention. Such an intervention may be, for example, a self-management intervention to be executed by the first user, and/or the intervention may be, for example, a natural support intervention to be executed by the second user for the benefit of the first user.

Block 406 includes delivering a prompt for the natural support intervention to at least one of the first user and a second user. For example, virtual mediation persona module 120 may send a prompt to perform a first intervention to user device 106 such that the first user is notified and encouraged to perform the first intervention. Examples of such a first intervention or care self-management may include but are not limited to initiating a chat or video conference session with the second user (who may be a family member, friend, doctor or caregiver), going for a walk, or performing some other task or activity expected to improve a state of the first user. In addition, or in the alternative, virtual mediation persona module 120 may send a prompt to perform the second intervention to user device 108 such that the second user is notified and encouraged to perform the second intervention for the benefit of the first user. Such a prompt may include and/or be delivered in a form of daily insights into the past or current state of the first user, for example a summary of prior and current emotional or physical states of the first user. Examples of such a second intervention may include but are not limited to initiating a chat or video conference session with the first user, who may be the patient, meeting up with the first user to socialize or to check in on his or her condition, or performing some other task or activity expected to improve the state of the first user.

Such prompted interventions, regular contact or communication for natural support intervention between the first and second users via user devices 106, 108 may be facilitated through the mediator, such that all electronic communications occur through system 110 and are thereby consolidated or aggregated.

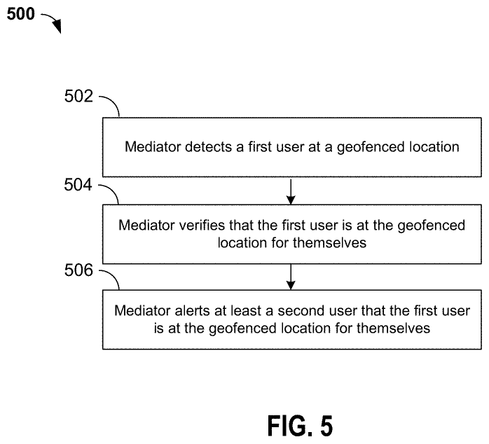

FIG. 5 illustrates a flowchart 500 of a method for selectively generating a geolocation-based natural support intervention for a user, in accordance with some embodiments. In some embodiments, the method of flowchart 500 may be performed by system 110, intervention mediator 102, or virtual mediation persona module 120, as previously described in connection with FIGS. 1A-2, any of which may be referred to as a mediator herein. In some embodiments, some or all of the described interactions with the first user and/or the second user may be mediated by VMP 606.

At block 502, the mediator detects a first user at a geofenced location. For example, global positioning system (GPS) functionality on user device 106 may provide a location of the first user to the mediator. Based on the received location, the mediator may determine that the location corresponds to or is associated with a health care facility (e.g., a clinic, urgent care, emergency room, hospital, or pharmacy) and thereby detect that the first user is at a geofenced location.

At block 504, the mediator verifies that the first user is at the geofenced location for themselves. For example, the mediator may send a message to user device 106 asking the first user about the detected location and prompting the first user to response via the app on user device 106. The mediator may perform a chat or other interaction with the first user via the app on user device 106 to confirm they are, indeed, at the detected geofenced location and that they are there for their own care, rather than for the care of another. If the first user indicates he or she is not at the detected geofenced location or that he or she is at the detected geofenced location but for the care of another, no further action may be taken in some cases.

At block 606, the mediator alerts at least a second user that the first user is at the geofenced location for themselves. For example, the mediator may send an alert (e.g., a text message) to user device 108 such that the second user may view a location alert history on the app (e.g., on a dashboard of the app) and is notified that the first user is at the geofenced location (e.g., the emergency room) for their own care. In some embodiments, the mediator may record the alert to the first user's dashboard such that a care manager (e.g., a doctor) may view the alert via their own dashboard on a smart phone, computer, or other electronic device.

Such detection, verification and alerting of the first user being at a geofenced location allows either or both of the first user's doctor or the second user (e.g., caregiver) to be notified in real-time or near real-time of the occurrence of a situation that could adversely affect the health of the first user and take appropriate preventative, or natural intervention support actions.

Several example screen-shots of an app configured to facilitate the above described natural support intervention for a first user will now be described in more detail in connection with FIGS. 6-13 below.

FIG. 6 illustrates several example screen-shots 602, 604 for an application configured to facilitate natural support intervention for a user, in accordance with some embodiments.

For example, screen-shot 602 illustrates an example dashboard within the app of either user devices 106, 108. Screen-shot 602 illustrates VMP 606 in the form of a robot avatar interacting with the user (e.g., saying “Welcome, John”). The dashboard may include but is not limited to links that allow the user to: access a user profile, manage a support network (e.g., the second user or “PyxPals”), view a level assigned to the user (e.g., “Space Explorer” based on a level, duration, or aggregate of prior interaction between the app and the user), and view health plan resources (e.g., “Find a Provider,” “Find a Dentist,” “Call Customer Care,” “Member Portal,” “Call Nurse Hotline,” or “Call Support”).

Screen-shot 604 illustrates an example including several levels potentially assignable to the user. For example, screen-shot 604 illustrates the ascending levels “New Cadet,” “Shooting Star,” “Astronaut Ally,” “Space Explorer,” “Solar Sidekick,” and “Cosmic Companion,” with the first 4 illustrated as having been achieved and the number of hours left to achieve each of the last 2 levels. Such potentially assignable levels provide entertainment, feedback and a sense of achievement associated with use of the app to the user, thereby improving compliance as well as the general state of the user.

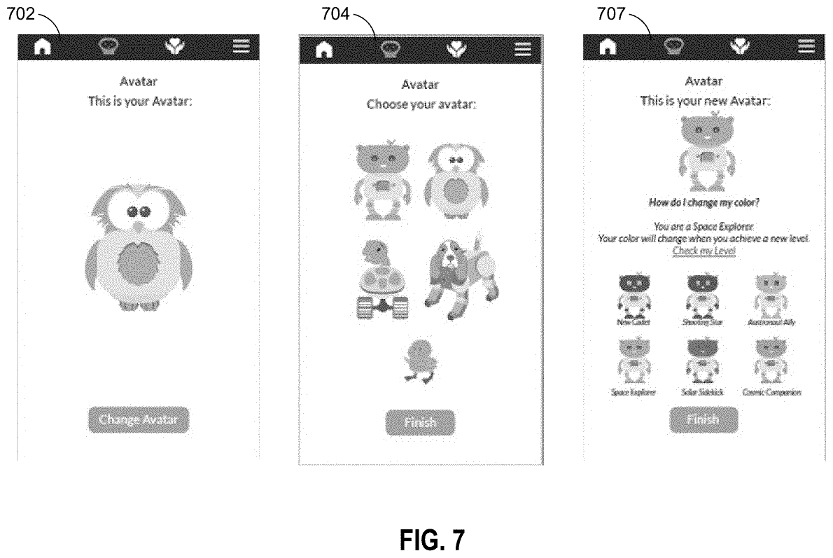

FIG. 7 illustrates several additional example screen-shots 702, 704, 706 for an application configured to facilitate natural support intervention for a user, in accordance with some embodiments.

For example, screen-shot 702 illustrates an avatar selected by the user (e.g., an illustration of an owl) as well as a button allowing the user to change their selected avatar. Screen-shot 704 illustrates a display for selecting an avatar from among a plurality of potential avatars, as well as a button allowing the user to complete their selection. Screen-shot 706 displays the user's selected avatar, notifies the user that one or more aspects of their avatar (e.g., color) may change as each new level is achieved, a link allowing the user to check their current level, as well as a button allowing the user to indicate they are finished with the current displayed page.

Allowing the user to customize their avatar as well as providing for one or more aspects of the avatar to change as new levels are reached provides both a sense of ownership to the user as well as a motivation to continue using the app in order to “level up.”

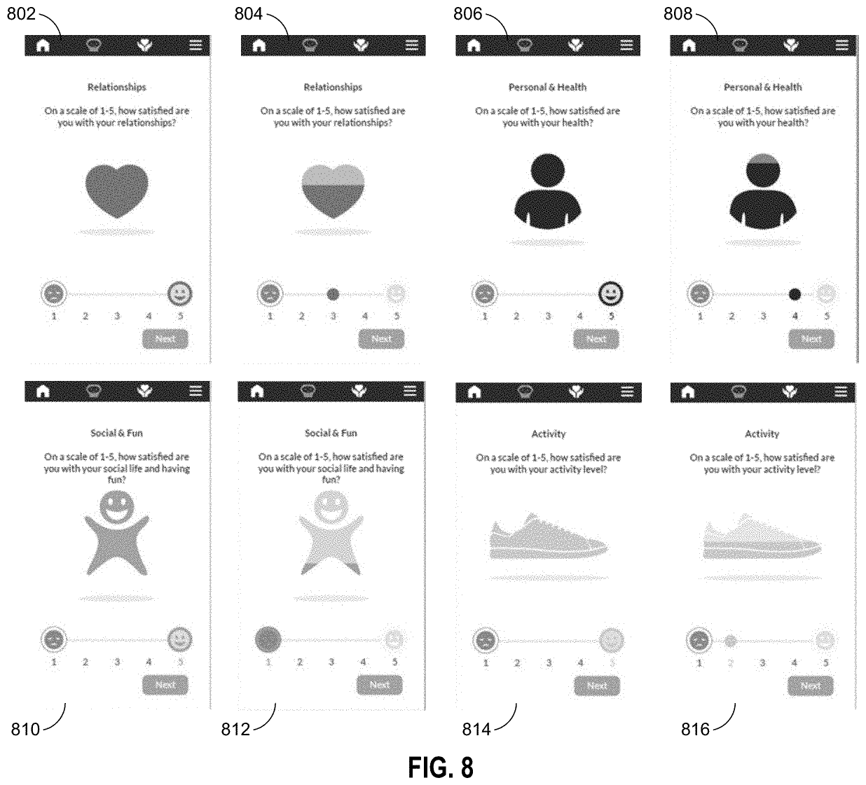

FIG. 8 illustrates several additional example screen-shots 802, 804, 806, 808, 810, 812, 814, 816 for an application configured to facilitate natural support intervention for a user, in accordance with some embodiments.

For example, screen-shot 802 illustrates the display of a question regarding the user's relationships “On a scale of 1-5, how satisfied are you with your relationships?”, an input (e.g., a virtual slider) that allows the user to interact with the app and/or VMP 606 and answer the question (e.g., by moving the slider to one of the notches between 1 and 5), and a button allowing the user to proceed to the next question. Screen-shot 802 also illustrates an image (e.g., a heart) that is substantially “full,” corresponding to a selection of 5 on the 1-5 scale slider. Screen-shot 804 is substantially the same as screen-shot 802, however, illustrating the image partially full, corresponding to a selection of 3 on the 1-5 scale slider.

Screen-shot 806 illustrates the display of a question regarding the user's personal health “On a scale of 1-5, how satisfied are you with your health?”, an input (e.g., a virtual slider) that allows the user to answer the question (e.g., by moving the slider to one of the notches between 1 and 5), and a button allowing the user to proceed to the next question. Screen-shot 806 also illustrates an image (e.g., a person's silhouette) that is substantially “full,” corresponding to a selection of 5 on the 1-5 scale slider. Screen-shot 808 is substantially the same as screen-shot 806, however, illustrating the image partially full, corresponding to a selection of 4 on the 1-5 scale slider.

Screen-shot 810 illustrates the display of a question regarding the user's social life “On a scale of 1-5, how satisfied are you with your social life and having fun?”, an input (e.g., a virtual slider) that allows the user to answer the question (e.g., by moving the slider to one of the notches between 1 and 5), and a button allowing the user to proceed to the next question. Screen-shot 810 also illustrates an image (e.g., a smiling figure jumping) that is substantially “full,” corresponding to a selection of 5 on the 1-5 scale. Screen-shot 812 is substantially the same as screen-shot 810, however, illustrating the image partially full, corresponding to a selection of 1 on the 1-5 scale.

Screen-shot 814 illustrates the display of a question regarding the user's activity “On a scale of 1-5, how satisfied are you with your activity level?”, an input (e.g., a virtual slider) that allows the user to answer the question (e.g., by moving the slider to one of the notches between 1 and 5), and a button allowing the user to proceed to the next question. Screen-shot 814 also illustrates an image (e.g., a shoe) that is substantially “full,” corresponding to a selection of 5 on the 1-5 scale slider. Screen-shot 816 is substantially the same as screen-shot 814, however, illustrating the image partially full, corresponding to a selection of 2 on the 1-5 scale slider.

Such an interface allows system 110 to provide questions 130 to the user and facilitates an entertaining, interactive, and fun way for the user to provide answers 132 with an increased level of engagement and compliance than might otherwise occur with less entertaining, interactive, and fun methods of delivery and interaction.

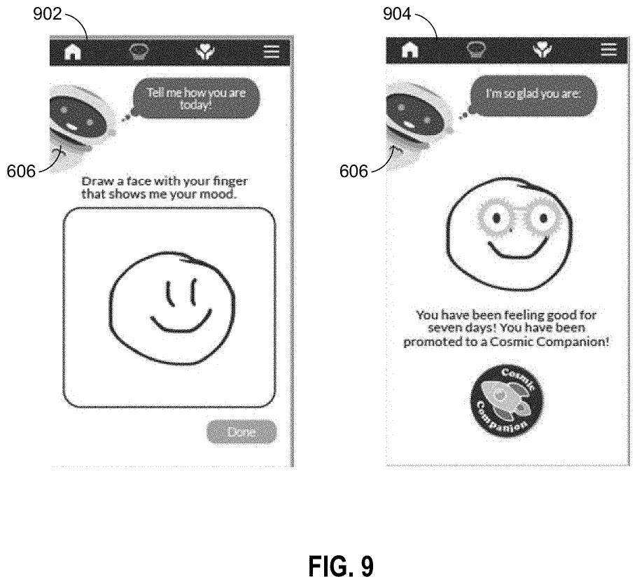

FIG. 9 illustrates several additional example screen-shots 902, 904 for an application configured to facilitate natural support intervention for a user, in accordance with some embodiments.

For example, screen-shot 902 illustrates VMP 606 interacting with the user “Tell me how you are today!”, and an area in which to “Draw a face with your finger that shows me your mood,” a smiley face illustrated as having been drawn in the area by the user, and a button allowing the user to proceed to the next question. Screen-shot 904 illustrates VMP 606 interacting with the user “I'm so glad you are:”, a modified version of the face drawn by the user (e.g., virtual mediation persona module 120 may be configured to add and/or adjust one or more features of the user drawing such as adding glasses to the eyes of the smiley face), and a historical summary indicating “You have been feeling good for seven days! You have been promoted to a Cosmic Companion!”, as well as an image corresponding to the newly achieved level.

Such drawing-based interaction with the user, and utilizing VMP 606, facilitates an entertaining, interactive, and fun way for the user to provide answers 132 with an increased level of engagement and compliance than might otherwise occur with less entertaining, interactive, and fun methods of delivery and interaction.

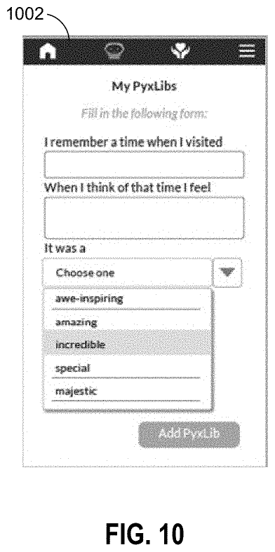

FIG. 10 illustrates an additional example screen-shot for an application configured to facilitate natural support intervention for a user, in accordance with some embodiments. For example, screen-shot 1002 illustrates a “mad libs” type interaction with the user that asks the user to “Fill in the following form:”, provides several statements (“I remember a time when I visited,” “When I think of that time I feel,” “It was a,”) for the user to complete and a button allowing the user to add the “PyxLib” to their profile.

Such “mad lib” type interactions with the user facilitate an entertaining, interactive, and fun way for the user to focus their attention on prior memories that are positive or empowering, which may allow for self-improvement of the emotional state of the user.

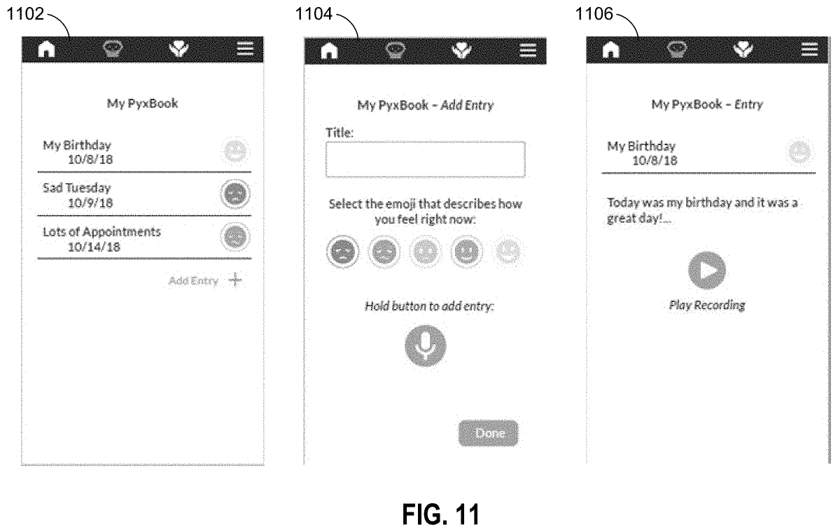

FIG. 11 illustrates several additional example screen-shots 1102, 1104, 1106 for an application configured to facilitate natural support intervention for a user, in accordance with some embodiments.

For example, screen-shot 1102 illustrates “My PyxBook”, which provides a library of prior daily emotions or states of the user (“My Birthday,” “Sad Tuesday,” “Lots of Appointments,”) and a link that allows the user to add another entry.

Screen-shot 1104 illustrates a page for entering a new entry in the library of daily emotions or states of the user, including a field for the user to enter a title, a plurality of emojis (or an emoji wheel for example) to select an appropriate emotional state, a microphone indicator to allow entry addition by speech, and a button to finish the entry.

Screen-shot 1106 illustrates a page summarizing the newly added entry from 1104, including the title, the selected emoji, a summary of the entry “Today was my birthday and it was a great day! . . . ,” and a button to play an associated recording from the user.

Such a library not only allows the mediator, as well as caregivers and other support users, to track the daily emotions and states of the user, it allows the user to go back and mentally revisit the good days, which may further enhance the current emotions and state of the users.

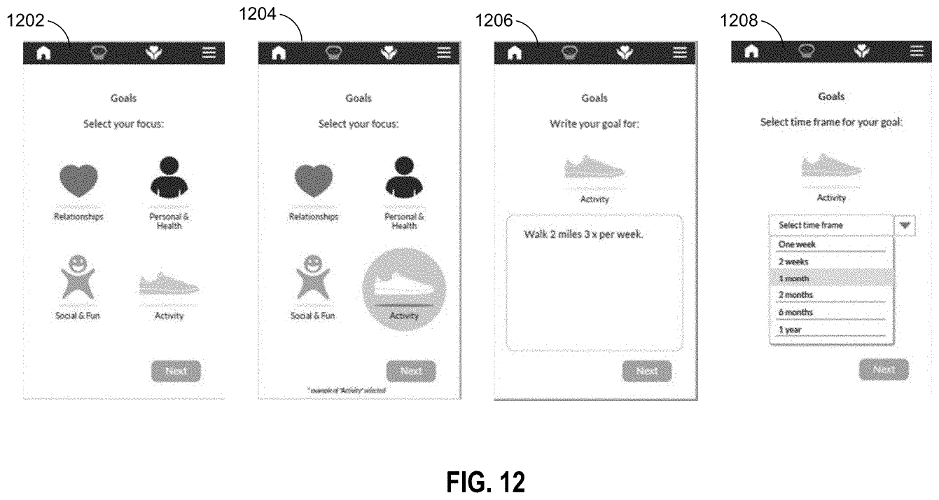

FIG. 12 illustrates several additional example screen-shots 1202, 1204, 1206, 1208 for an application configured to facilitate natural support intervention for a user, in accordance with some embodiments.

For example, screen-shot 1202 illustrates a screen for the user to enter one or more goals according to a particular focus: “Select your focus,” an image or button associated with each of a plurality of focuses (e.g., Relationships, Personal & Health, Social & Fun, and Activity”), and a button allowing the user to proceed to the next screen. Screen-shot 1204 is substantially the same as screen-shot 1202 except illustrating “Activity” as being selected.

Screen-shot 1206 illustrates a page for entering a goal specific to the selected focus of “Activity”: “Walk 2 miles 3× per week,” and having a button to finish the entry. Screen-shot 1208 illustrates a page for entering or selecting a time frame for the specific goal.

Facilitating such personal goal setting in a variety of areas of focus not only allows the user to self-manage their care in a variety of dimensions that affect heath, but allows the user to feel self-directed in that they are able to both set the specifics of the goal themselves, as well as the timeframe during which they will strive to achieve that goal. This may provide a feeling of control to the user that increases their motivation and compliance with their self-directed goals and health-plan.

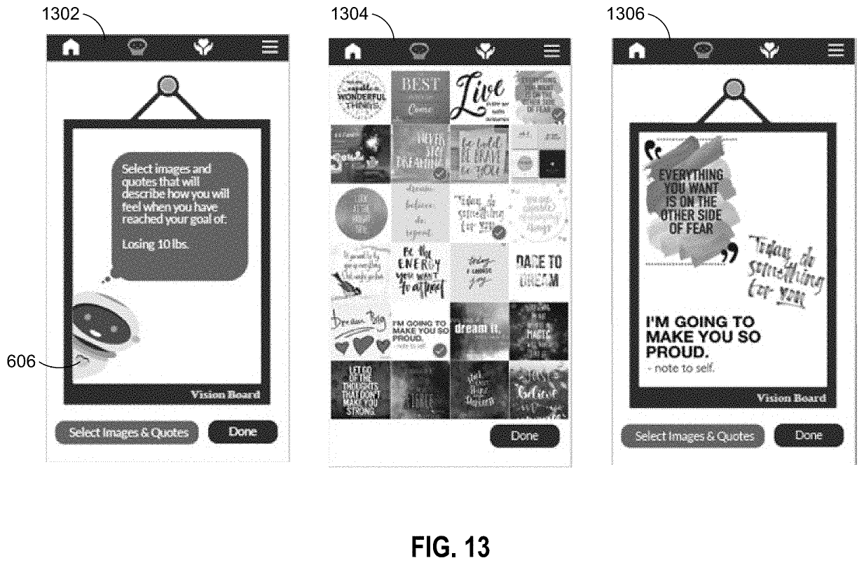

FIG. 13 illustrates several additional example screen-shots 1302, 1304, 1306 for an application configured to facilitate natural support intervention for a user, in accordance with some embodiments.

For example, screen-shot 1302 illustrates a screen for the user to create a vision board comprising a plurality of images, quotes or sayings designed to instill good emotions in the user. VMP 606 is illustrated as prompting the user to “Select images and quotes that will describe how you will feel when you have reached your goal of: Losing 10 lbs,” as well as a button allowing the user to select images and quotes from a library and a button allowing the user to proceed when done. Screen-shot 1304 illustrates a plurality of thumbnail images of the images and quotes available for selection by the user. Screen-shot 1306 illustrates a generated vision board including images selected by the user, as well as a button allowing the user to select further images and quotes from the library and a button allowing the user to proceed when done.

Facilitating the self-generation of such a vision board by the user allows the user to focus on the positive emotions they will experience when they reach their goal, thereby providing a positive state and experience to the user daily as they focus on achieving their goal day-to-day.

System 110, user device 106 and user device 108 are operational with numerous general-purpose or special-purpose computing system environments, configurations, processors and/or microprocessors. Examples of well-known computing systems, environments, and/or configurations that may be suitable for use with the technology disclosed herein include, but are not limited to, personal computers, server computers, hand-held or laptop devices, multiprocessor systems, microprocessor-based systems, programmable consumer electronics, network PCs, minicomputers, mainframe computers, distributed computing environments that include any of the above systems or devices, and the like.

A microprocessor may be any conventional general-purpose single- or multi-chip microprocessor such as but not limited to a Pentium® processor, a Pentium® Pro processor, a 8051 processor, a MIPS® processor, a Power PC® processor, or an Alpha® processor. In addition, the microprocessor may be any conventional special purpose microprocessor such as a digital signal processor or a graphics processor. The microprocessor typically has conventional address lines, conventional data lines, and one or more conventional control lines.

System 110 comprises various modules/components as discussed in detail above. As can be appreciated by one of ordinary skill in the art, each of the modules comprises various sub-routines, procedures, definitional statements and/or macros. Since functionality of one module may be performed along with or by one or more other modules, the description of each of the modules is used for convenience to describe the functionality of the preferred system. Thus, the processes that are undergone by each of the modules may be arbitrarily redistributed to one of the other modules, combined together in a single module, or made available in, for example, a shareable dynamic link library.

System 110 may be used in connection with various operating systems such as but not limited to Linux®, UNIX® or Microsoft Windows®. Instructions or code utilized by or for system 110 may be written in any conventional programming language such as but not limited to C, C++, BASIC, Pascal, or Java, and ran under a conventional operating system.

A web browser comprising a web browser user interface may be used to display information (such as textual and graphical information) to a user as described above. The web browser may comprise any type of visual display capable of displaying information received via a network. Examples of web browsers include but are not limited to Microsoft's Internet Explorer browser, Netscape's Navigator browser, Mozilla's Firefox browser, PalmSource's Web Browser, Apple's Safari, or any other browsing or other application software capable of communicating with a network.

In one or more example embodiments, the functions and methods described may be implemented in hardware, software, or firmware executed on a processor, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Further, it should be appreciated that modules and/or other appropriate means for performing the methods and techniques described herein can be downloaded and/or otherwise obtained by a user device/terminal and/or base station as applicable. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

While the foregoing is directed to aspects of the present disclosure, other and further aspects of the disclosure may be devised without departing from the basic scope thereof, and the scope thereof is determined by the claims that follow.

What is claimed is:

1. A system for aggregating communication channels to generate effective interventions, the system comprising:
an intervention mediator configured to electronically communicate with at least one of a first user via a first user device and a second user via a second user device, the mediation module configured to:
assess whether an intervention with the first user is appropriate;
determine a specific intervention based at least in part on a result of the assessment; and
provide the second user, via the second user device, a prompt to perform the specific intervention for the benefit of the first user.

2. The system of claim 1, wherein the intervention mediator is configured to:
generate a score based at least in part on feedback from the first user via the first user device;
determine the specific intervention by mapping the score to the specific intervention.

3. The system of claim 1, wherein the intervention mediator is configured to:
generate, for display on the first user device, a first virtual mediation persona configured to:
present to the first user a plurality of questions for assessing whether the intervention for the first user is appropriate;
receive from the first user via the first user device a plurality of answers to the plurality of questions; and
provide an interaction with the first user via the first user device based on the plurality of answers.

4. The system of claim 3, wherein the intervention mediator is configured to generate, for display on the second user device, a second virtual mediation persona configured to notify the second user of at least one of the plurality of answers to the plurality of questions from the first user.

5. The system of claim 3, wherein the intervention mediator is configured to generate, for display on the second user device, a second virtual media persona configured to prompt the second user to interact with the first user via the system.

6. The system of claim 3, wherein the intervention mediator is configured to:
perform a screening of an emotional state of the first user based on at least one of the plurality of answers from the first user; and
provide to the first user, via the first user device, at least one resource usable by the first user to self-manage the emotional state based on the screening.

7. The system of claim 3, wherein the intervention mediator is configured to:
perform a screening of at least one social determinant of health of the first user based on at least one of the plurality of answers from the first user; and
provide to the first user, via the first user device, at least one resource usable by the first user to self-manage the social determinant of health based on the screening.

8. The system of claim 1, wherein the intervention mediator is configured to:
detect the first user at a geofenced location;
verify that the first user is at the geofenced location for their own care; and
alert at least the second user via the second user device that the first user is at the geofenced location for their own care.

9. The system of claim 8, wherein the intervention mediator is configured to generate, for display on the first user device, a first virtual media persona configured to ask the first user at least one question to verify that the first user is at the geofenced location for their own care.

10. The system of claim 1, further comprising a communication module configured to aggregate a plurality of communication channels within the system, each channel providing communication between at least two of the intervention mediator, the first user device, and the second user device.

11. A method for selectively generating a natural support intervention, comprising:
assessing whether a natural support intervention for a first user is appropriate;
determining an appropriate natural support intervention based at least in part on a result of the assessment; and
prompting at least one of the first user, via a first user device, and a second user, via a second user device, to perform the appropriate natural support intervention for the benefit of the first user.

12. The method of claim 11, comprising:
generating a score based at least in part on feedback from the first user via the first user device;
mapping the score to the appropriate natural support intervention; and
delivering a prompt for the appropriate natural support intervention to at least one of the first user, via the first user device, and the second user, via the second user device.

13. The method of claim 11, comprising generating, for display on the first user device, a first virtual media persona configured to:
present to the first user a plurality of questions for assessing whether the natural support intervention for the first user is appropriate;
receive from the first user via the first user device a plurality of answers to the plurality of questions; and
provide an interaction with the first user via the first user device based on the plurality of answers.

14. The method of claim 13, comprising generating, for display on the second user device, a second virtual media persona configured to notify the second user of at least one of the plurality of answers to the plurality of questions from the first user.

15. The method of claim 13, comprising generating, for display on the second user device, a second virtual media persona configured to prompt the second user to interact with the first user.

16. The method of claim 13, comprising:
performing a screening of an emotional state of the first user based on at least one of the plurality of answers from the first user; and
providing to the first user, via the first user device, at least one resource usable by the first user to self-manage the emotional state based on the screening.

17. The method of claim 13, comprising:
performing a screening of at least one social determinant of health of the first user based on at least one of the plurality of answers from the first user; and
providing to the first user, via the first user device, at least one resource usable by the first user to self-manage the social determinant of health based on the screening.

18. The method of claim 11, comprising:
detecting the first user at a geofenced location;
verifying that the first user is at the geofenced location for their own care; and
alerting at least the second user via the second user device that the first user is at the geofenced location for their own care.

19. The method of claim 18, comprising generating, for display on the first user device, a first virtual media persona configured to ask the first user at least one question sufficient to verify that the first user is at the geofenced location for their own care.

20. The method of claim 11, comprising aggregating a plurality of communication channels between at least two of the first user device, the second user device, and a system utilized to facilitate communication between the first user and the second user.
